(19) United States
(12) Patent Application Publication
(10) Pub. No.: US 2004/0186828 A1
(43) Pub. Date: Sep. 23, 2004

(54) Systems and methods for enabling a user to find information of interest to the user

(76) Inventors: Prem Yadav, Oak Hill, VA (US); Ken Wasserman, Frederick, MD (US); Vadim L. Ravich, Clifton, VA (US); Tony Piselli, Mauertown, VA (US); Truc Trung Nguyen, Manassas, VA (US); Ashit Kumar, South Riding, VA (US); Jay George, Gaithersburg, MD (US); Yan Ding, Herndon, VA (US)

Correspondence Address: ROTHWELL, FIGG, ERNST & MANBECK, P.C., 1425 K STREET, N.W., SUITE 800, WASHINGTON, DC, 20005, US

(21) Appl. No.: 10/743,322

(22) Filed: Dec. 23, 2003

Related U.S. Application Data
(60) Provisional application No. 60/435,870, filed on Dec. 24, 2002.

(51) Int. Cl. G06F 17/30

(52) U.S. Cl. 707/3

(57) ABSTRACT

The present invention provides users with access to Internet-accessible databases via one portal of entry, such that queries need not be repeated multiple times in order to obtain needed information. Advantageously, the present invention will harness a systematic dynamic query profiler, document scoring, and display of retrieved documents via a knowledge-based system that facilitates user editing. Thus, the present invention will aid users so that less of their time and effort are required in order to obtain precisely the desired information for which they are searching.

### CROSS REFERENCE TO RELATED APPLICATION

This application claims the benefit of U.S. Provisional Patent Application No. 60/435,870, filed on Dec. 24, 2002, the contents of which are incorporated herein by reference.

### BACKGROUND OF THE INVENTION

1. Field of the Invention

The present invention relates to systems and methods for enabling a user to find information of interest to the user, and, in one embodiment, to an automatic information retrieval system for finding project specific, scientific information from information sources accessible via the Internet. The automatic information retrieval system is referred to herein as: Xactans™ (which stands for exact-answer).

2. Discussion of the Background

Recent years have seen explosive growth in the number and content of vital, biological databases, which contain essential information regarding structural biology, genomics, proteomics, metabolic and signal transduction pathways, clinical trial results, chemical structures, and Patents-both applied and granted. The ability of the scientific community to access this essential information relies almost completely upon well-established search engines, such as PubMed Central, the US Patent and Trademark Office (USPTO) patent databases, and Google™. Many individual publishers have designed their own search engines such as Elsevier Sciences ScienceDirect, and Wiley InterSciences service, but these are of extremely limited scope.

Unfortunately, a user-friendly search engine capable of providing a single portal with sufficient reach to provide desired information to the research community has yet to be introduced. Moreover, we have recently learned that the Department of Energy shut down the public domain resource “PubScience” that cross indexed nearly 2 million government reports and academic articles.

Another disadvantage of conventional private scientific search engines, such as SciRus, SciFinder®, and Search4Science, which access online resources and their own databases, is that they cannot be customized based on site licenses of user institutions or individual subscribers.

Additionally, the most commonly used search engines provide access to only a fraction of the desired information. For example, to obtain basic information regarding genomes, primary nucleotide, or amino acid sequence and protein structural data, a user might query National Center for Biotechnology Information (NCBI) databases. However, a more informed user might also query other databases: e.g. the Stanford Microarray database, PlasmoDB at the University of Pennsylvania, the metabolic pathway database at Yale University, Structural Classification of Protein (SCOP) at Cambridge, UK, the Nucleic Acid Data Bank (NDB) and Protein Data Bank (PDB) at Rutgers University, Signaling Pathway database (SPAD) and DNA database of Japan, the Transgenic and Targeted Mutant Animal Database (TBASE) at John Hopkins, Clintrials clinical studies database, and the USPTO databases—just to name a few.

Existing autonomous, biological databases contain related data that are more valuable when interconnected. However, it is currently not possible to simultaneously query related data because source databases are built by different teams, in different locations, for different purposes, and are comprised of different database architectures and design. To obtain desired information, rigorous scientists must query multiple remote or local heterogeneous data sources, and manually integrate retrieved data without the aid of intelligent data analysis and visualization tools.

Currently available search engines typically input keywords or phrases as well as Boolean logic terms such as “AND”, “NOT” and “OR” to logically connect the keywords/phrases. Such search engines can monitor and rank query output based on hit frequencies or chronology, such that more recent database inputs, or popular links, as determined by the user community, appear first in a query output list. Output can also appear ranked by one or more hyperlink patterns, independent of precise search specifications. This is based on the assumption that important web pages are likely to be those that have relatively numerous links to other pages, or are frequently linked from other pages.

Unfortunately, current ranking schemes often provide the desired output mixed in with a great deal of undesired output. Thus, users must scan query output manually to find what they need.

Another drawback of conventional search systems is that they do not enable a user to maintain current and updated information regarding topics of interest. Moreover, scientific investigators have aligned themselves into specialized areas, and might benefit from a search engine capable of enlarging their peripheral vision.

What is desired, therefore, are search systems and methods to overcome the above described and other disadvantages of the conventional search system and methods.

### SUMMARY OF THE INVENTION

In one aspect, the present invention provides users with access to Internet-accessible databases via one portal of entry, such that queries need not be repeated multiple times in order to obtain needed information. Advantageously, the present invention will harness a systematic dynamic query profiler, document scoring, and display of retrieved documents via a knowledge-based system that facilitates user editing. Thus, the present invention will aid users so that less of their time and effort are required in order to obtain precisely the desired information for which they are searching. Because queries are repeated over time by a user, the present invention offers the users the ability to maintain a search profile and/or the results of past queries in their own datastore, in private accounts.

In short, the present invention provides information retrieval systems and methods. The computer systems and computer implemented methods of the present invention overcome the above described and other disadvantages of the conventional systems and methods.

In one embodiment, the computer implemented method of the present invention enables a user to easily find and retrieve the information of interest to user, and includes the following steps: prompting a user to input an initial query and receiving the initial query input by the user, wherein the initial query includes a keyword; determining a synonym of the keyword; determining a term related to the keyword; creating a first query, wherein the first query (a) includes the keyword, the synonym, and/or the related term and (b) conforms to the query protocol of a first search engine; creating a second query, wherein the second query (a) includes the keyword, the synonym, and/or the related term and (b) conforms to the query protocol of a second search engine; submitting to the first search engine the first query; submitting to the second search engine the second query; receiving from the first search engine a first plurality of document identifiers; receiving from the second search engine a second plurality of document identifies; and for one or more document identifier included in the first plurality of document identifiers and for one or more document identifier included in the second plurality of document identifiers, determining a score for the document identified by the document identifier, wherein the step of determining the score includes the step of identifying a figure legend within the document, and wherein the document's score is, at the least, a function of whether the keyword, synonym and/or related word is found in the identified figure legend.

Advantageously, a network of adaptable scoring matrices is created and used in scoring a document. The scoring matrices can have 1, 2, 3 or N dimensions. For example, in one embodiment, a 2 dimensional scoring matrix relating the number of keywords in a document's abstract with the number of related terms in the abstract can be used.

In another aspect, the present invention includes a computer readable medium, such as, for example, an optical or magnetic data storage device, having stored thereon software for implementing the methods of the invention.

The above and other features and advantages of the present invention, as well as the structure and operation of preferred embodiments of the present invention, are described in detail below with reference to the accompanying drawings.

### BRIEF DESCRIPTION OF THE DRAWINGS

The accompanying drawings, which are incorporated herein and form part of the specification, illustrate various embodiments of the present invention and, together with the description, further serve to explain the principles of the invention and to enable a person skilled in the pertinent art to make and use the invention. In the drawings, like reference numbers indicate identical or functionally similar elements. Additionally, the left-most digit(s) of a reference number identifies the drawing in which the reference number first appears.

FIG. 1 is a functional block diagram of a system according to an embodiment of the present invention.

FIGS. 2A-B show a flow chart illustrating a process according to an embodiment of the present invention.

FIG. 3 illustrates an example user interface that enables a user of the system to select one or more databases to search and to input a query into the system.

FIG. 4 illustrates an example user interface that enables the user to create an enhanced query.

FIG. 5 is a flow chart illustrating a process according to an embodiment of the present invention.

FIG. 6 shows a representative database table for storing document information.

FIG. 7 illustrates examples scoring matrices of the present invention.

FIG. 8 illustrates an example network of scoring matrices.

FIG. 9 illustrates an example list of documents outputted by the system.

FIG. 10 is an illustration of a representative computer system that can be used to implement the systems and methods of the present invention.

### DETAILED DESCRIPTION OF THE PREFERRED EMBODMENT

In the following description, for purposes of explanation and not limitation, specific details are set forth, such as particular systems, computers, devices, components, techniques, computer languages, storage techniques, software products and systems, operating systems, interfaces, hardware, etc. in order to provide a thorough understanding of the present invention. However, it will be apparent to one skilled in the art that the present invention may be practiced in other embodiments that depart from these specific details. Detailed descriptions of well-known systems, computers, devices, components, techniques, computer languages, storage techniques, software products and systems, operating systems, interfaces, and hardware are omitted so as not to obscure the description of the present invention.

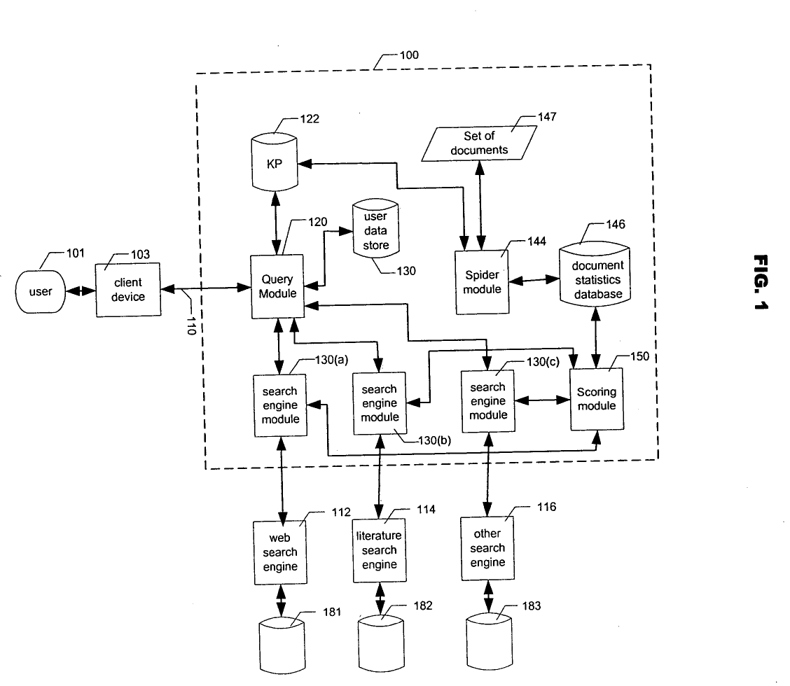

The present invention provides an automatic information retrieval system 100 (see FIG. 1), which is referred to herein as Xactans 100. Xactans 100 can be used to retrieve information pertaining to any subject area or profession, such as, for example, medical information, legal information, engineering information. For the purpose of illustration, and not limitation, a single application of Xactans 100 will be described herein. More specifically, we will describe how Xactans 100 can be used to retrieve and sort information pertaining to the life sciences.

A user 101 who is searching for information on a life sciences topic, but who may or may not be a subscriber of Xactans 100, may submit a query to Xactans 100. User 101 may use a client device 103 (e.g., a personal computer, mobile phone, personal digital assistant, or other communication capable device) to submit the query to Xactans 100 via the Internet 110 or other network (or the Xactans system may be locally stored on user 101's device 103). The query must include at least one string of characters (e.g., letters, numbers or other characters). If the query includes more than one string of characters the strings can be combined using, for example, boolean operators, such as, “AND” and “OR”.

After user 101 submits a query to Xactans 100, Xactans 100 may submit one or more queries to one or more web search engines 112 (e.g., Google™), which have access to documents available via the world-wide-web (WWW) 181, one or more search engines 114 for a database containing information related to the life sciences (e.g., PubMed Central and Scirus) 182, and/or a search engine 116 for one or more other databases 183 that may contain information related to the life sciences (e.g., the USPTO patent database, sequence databases, clinical trial databases, etc.). The one or more queries are identical to or based, at least in part, on the query submitted by user 101. Xactans 100 then analyzes and scores the responses from the search engines and provides information to user 101. Preferably, the information is information that user 101 was looking for. The information provided to user 101 may include a list of links to documents, a list of document titles, etc.

In the manner described above, Xactans 100 provides a user with access to network accessible databases via one portal of entry, such that queries need not be repeated multiple times in order for the user to obtain the desired information. In some embodiments, Xactans 100 includes a module that will present the information provided to the user in such a way that less user time and effort is needed in order for the user to obtain precisely the information for which the user was searching. As used herein, the term module means a set of computer instructions.

Additionally, in some embodiments, Xactans 100 offers users the ability to maintain their own datastore, in private accounts, that contain information retrieved by Xactans 100, and Xactans 100 may also enable user to more easily encounter supplemental information of direct relevance to their original query.

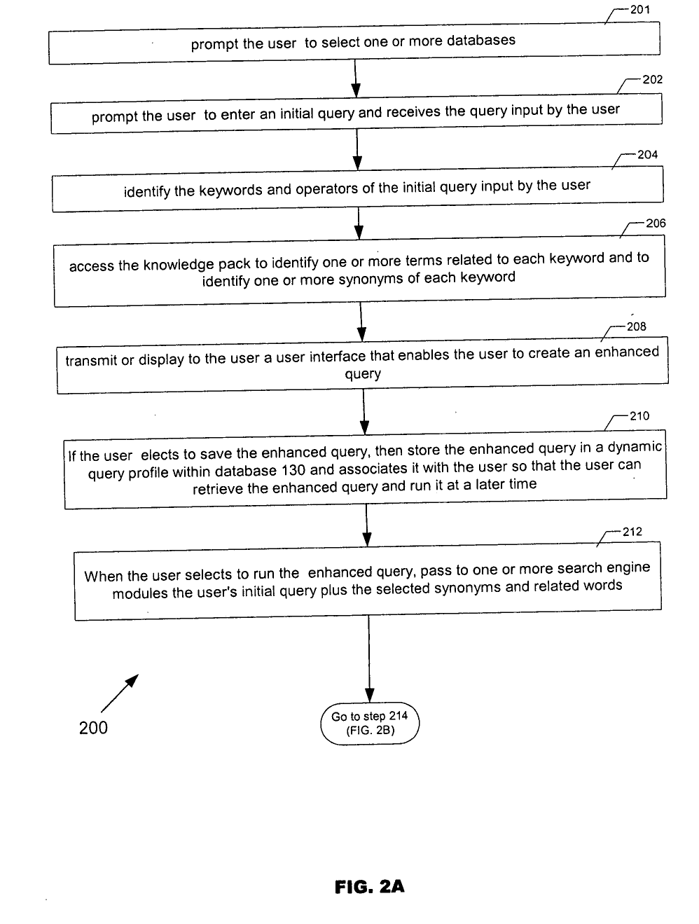

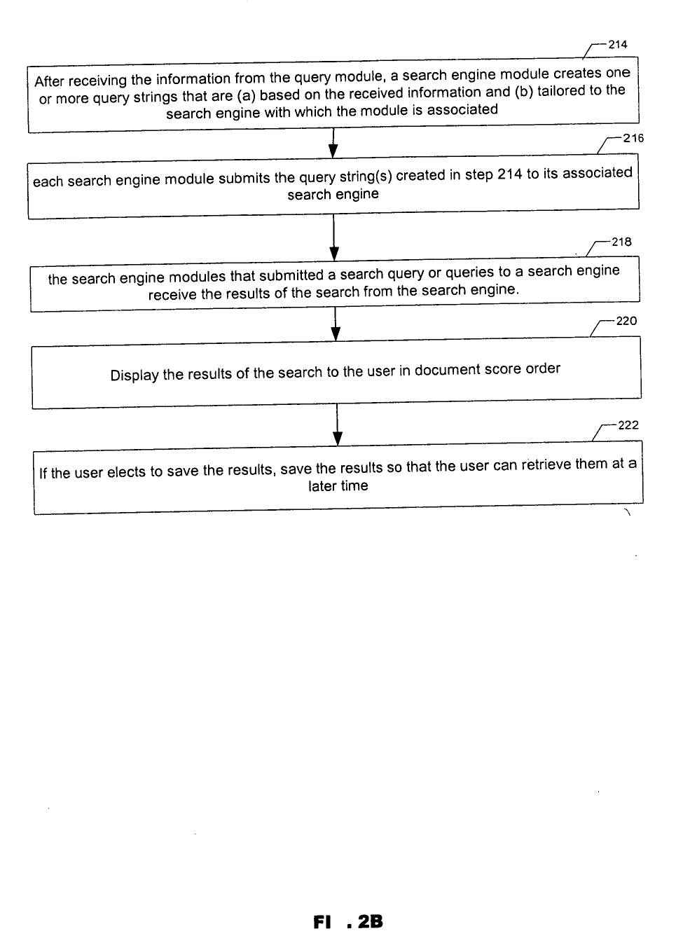

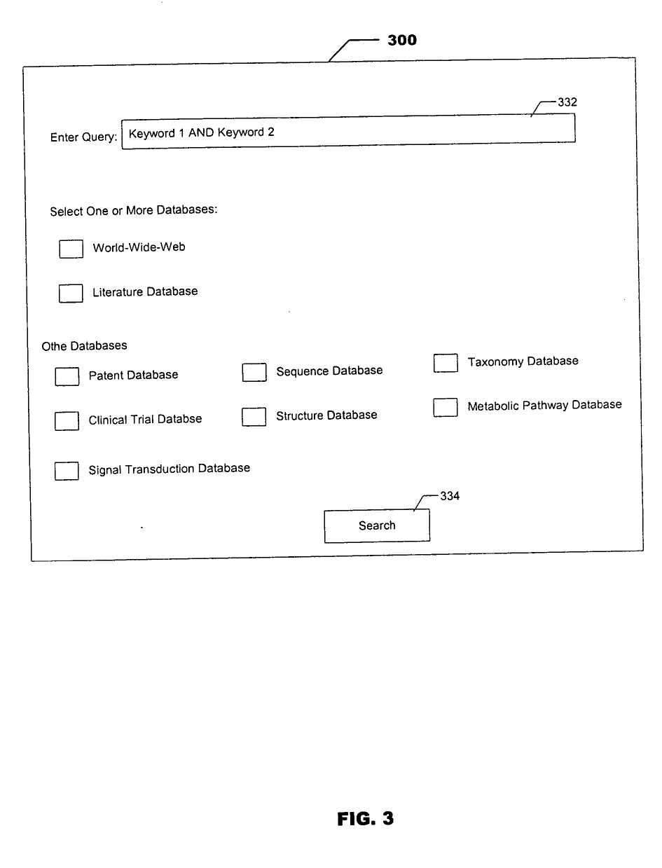

As shown in FIG. 1, Xactans 100 may include a query module 120, which is configured to interact with user 101. A process 200 that may be performed, at least in part, by query module 120 in some applications of the invention is illustrated in the flowchart shown in FIGS. 2A-B. As shown in FIG. 2A, process 200 may begin in step 201, where query module 120 prompts user 101 to select the databases to be searched. For example, in step 201, query module 120 may transmit or display to user 101 a user interface 300 (see FIG. 3), which enables user 101 to select one or more databases. User interface 300 is an example user interface that may be used in the embodiments where Xactans 100 is used to find life-sciences information, as opposed to other embodiments where Xactans 100 is used to find legal information or information in the field of engineering. As such, user interface 300 allows user 101 to select to search the WWW 181, a database containing life-science journal articles (e.g., literature database 182), and/or specialized databases 183 containing information related to a subject area within life-sciences. After user 101 makes his/her selection, process 200 may proceed to step 202.

In step 202, query module 120 prompts user 101 to enter an initial query and receives the query input by user 101. For example, interface 300 may include a field 332 into which a user can enter an initial query. In this example, user 101 submits the entered query to query module 120 by activating a “search” button 334.

After performing step 202, query module 120 identifies the keywords and operators of the initial query input by user 101 (step 204). For example, if the user submitted the following initial query: “‘reverse transcriptase’ AND ‘HIV’”, then query module 120 would identify “reverse transcriptase” and “HIV” as the two keywords and “AND” as an operator that links the two keywords.

Next (step 206), query module 120 accesses a knowledge pack (a.k.a., “KP”) 122 component of Xactans 100 to identify one or more terms related to each keyword and to identify one or more synonyms of each keyword. The knowledge pack 122, in this embodiment, is a database of terms (i.e., words or phrases) related to the life sciences (in other embodiments, for example where Xactans 100 is used for retrieving legal information, the knowledge pack 122 may contain legal terms). Each term (i.e., word or phrase) in the database 122 is associated with the term's synonyms and related terms. Thus, the knowledge pack 122 is like a thesaurus. Accordingly, if a keyword entered by user 101 matches a term in the knowledge pack 122, then query module 120 can obtain synonyms and related terms for the keyword by searching the knowledge pack database 122 for the keyword and then retrieving from the database the associated synonyms and related words. In this embodiment, the knowledge pack includes concept names from the Unified Medical Language System (UMLS). An administrator of Xactans 100 may add user defined terms to the knowledge pack. We expect that knowledge pack 122 will grow over time.

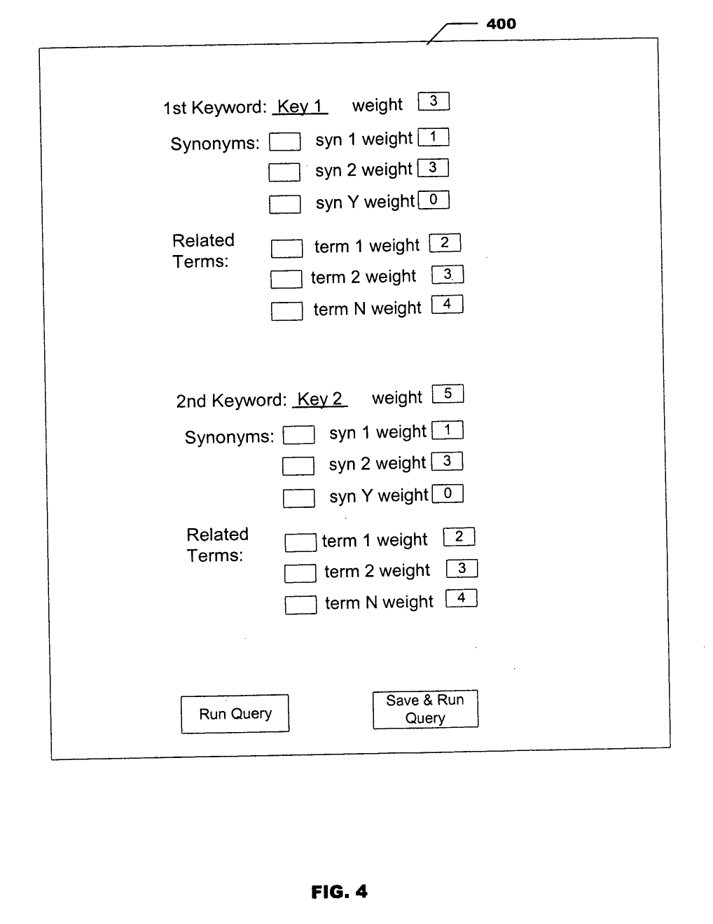

In step 208, query module 120 transmits or displays to user 101 a user interface 400 (see FIG. 4) that enables user 101 to create an enhanced query. That is, the user interface 400 is configured to display, for each identified keyword, a set of synonyms of the keyword and a set of terms related to the keyword.

User interface 400 allows a user to select one or more of the displayed synonyms and/or one or more of the listed related terms. Additionally, as shown in FIG. 4, interface 400 includes pull-down selection boxes that enable user 101 to assign a weight value to a displayed keyword, synonym and/or related term.

After user 101 makes his/her selections (i.e., selects zero or more synonyms and/or related terms and specifies weight values), user 101 may save the enhanced query (i.e., the keywords and selected synonyms, related terms and weights) and/or run the search. If user 101 elects to save the enhanced query, then query module 120 stores the enhanced query in a dynamic query profile within a database 130 and associates it with user 101 so that user 101 can retrieve it and run it at a later time (step 210). Preferably, user 101 gives each enhanced query a unique name prior to the enhanced query being stored in the database 130 so that database 130 can store more than one enhanced query associated with user 101.

When user 101 selects to run an enhanced query, query module 120 passes to one or more search engine modules 130 user 101's initial query plus the selected synonyms and related words (step 212). Each module 130 is associated with a different search engine. For example, module 130(a) may be associated with Google, module 130(b) may be associated with PubMed and module 130(c) may be associated with the USPTO patent database. More specifically, query module 120 passes the initial query plus the selected synonyms and related words to a search engine module only if the module is associated with one of the databases that user 101 selected using interface 300.

After receiving the information from query module 120, a module 130 creates one or more query strings that are (a) based on the received information and (b) tailored to the search engine with which the module is associated (step 214). For example, assume that query module 120 sent to module 130(b) user 101's initial query and user 101's selected synonyms and related terms; in this case module 130(b) may create a query that includes all of the keywords entered by user 101 and all of the synonyms and related terms selected by user 101. More specifically, the synonyms and related words selected for a given keyword are combined with the keyword using the Boolean “OR” operator.

For example, if user 101's initial query was: “key1 AND key2” and user 101 selected one synonym for key1 (e.g., syn1) and one related term for key2 (e.g., rt1), then the query created by module 103(b) may look as follows: “(key1 OR syn1) AND (Key2 OR rt1)”. However, if the search engine with which module 130(b) is associated can not process the “OR” operator, then module 130(b) may create four query strings: (1) “Key1 AND Key2”; (2) “Key1 AND rt1”; (3) “syn1 AND Key2”; and (4) “syn1 AND rt1” for that search engine.

Next (step 216), each module 130 submits the query string(s) created in step 214 to its associated search engine. For example, if user 101 selected to search the WWW 181, then module 130(a) submits the query string(s) created in step 214 to the WWW search 112 engine, such as, for example Google. As mentioned above, module 130(a) creates query strings that are tailored to the search engine that it uses. It does this so that the search engine can parse the query without errors. That is, in the example given, the query string submitted to Google conforms to the Google protocol for query strings. Similarly, if user 101 selected to search a database of journal articles, then module 130(b) submits the query string(s) created in step 214 to, for example, the PubMed Central search engine 114.

Next (step 218), the modules 130 that submitted a search query or queries to a search engine receive the results of the search. Typically, the results include a list of document identifiers (e.g., a list of hyperlinks each of which points to a document that matched the search, a list of document titles, etc.). The lists or combined lists are then displayed to user 101 (step 220).

In one embodiment, the results are displayed in the order received. Thus, in this embodiment, Xactans 100 does not rank the documents. However, in a preferred embodiment, Xactans 100 scores each document identified in the results and displays the list of document identifiers in rank order with the highest scoring documents being at the top of the list.

In one embodiment, a document's score is a function of: (a) the frequency with which each query term (i.e., keyword, synonym and related term) is found in the document (hereafter “query term frequency”); and (b) the weights associated with each query term.

In another embodiment, a document's score is a function of: (a) whether or not a query term is found in the document's title; (b) whether or not a query term is found in a figure legend of the document; (c) the frequency with which each query term is found in the document's abstract (“query term abstract frequency”); (d) the frequency with which each query term is found in the document's main body (“query term main body frequency”); and (e) the weights associated with the query terms.

In one embodiment, Xactans 100 determines the frequencies mentioned above after the modules 130 receive the search results from the search engines to which they submitted the queries. For example, after a module 130 submits a query string to a search engine and receives the list of document identifiers from the search engine, the module 130 may retrieve all of the identified documents and then parse the documents to determine the frequencies. It may also parse a document to find the documents title and all of its figure legends and to determine whether or not a query term is included in the title and/or figure legend. After determining the frequencies for a document, the frequency information may be provided to a scoring module 150, which uses the information to determine a score for the document.

In other embodiments, Xactans 100 determines the frequencies, for at least some of the identified documents, using information from a document database 146. Preferably, database 146 is created and populated with relevant information prior to user 101 entering the initial query. In these embodiments, in addition to including document database 146, Xactans 100 includes a spider module 144, which, preferably, has complete access to a large set of documents 147 (e.g., the set of documents to which the PubMed search engine has access among others). Spider 144 is configured to populate the database with information that enables Xactans 100 to determine; the query term frequency, query term abstract frequency, query term main body frequency, whether a certain term appears in a documents title, and whether a certain term appears in a figure legend.

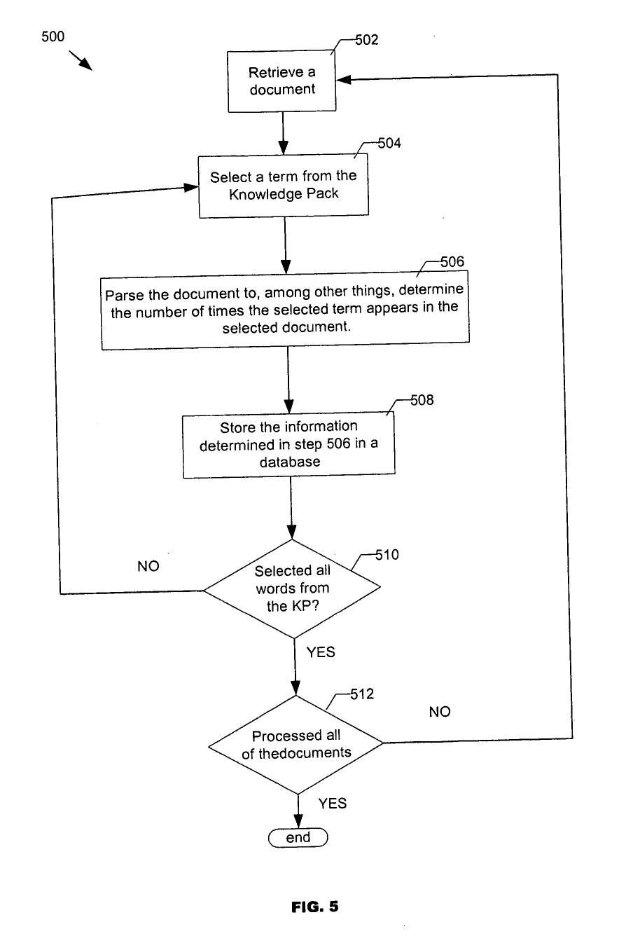

FIG. 5 is a flow chart illustrating a process 500 performed by spider 144. Process 500 may begin in step 502, where spider 144 retrieves a document from the set of documents. In step 504, spider 144 selects a word or term from the knowledge pack 122. In step 506, spider 144 parses the document to determine: (a) whether the word or term appears in the documents title; (b) whether the word or term appears in any figure legends; (c) whether the document has an abstract and, if so, the frequency with which the word or term appears in the abstract; and (d) the frequency with which the word or term appears in the main body of the document.

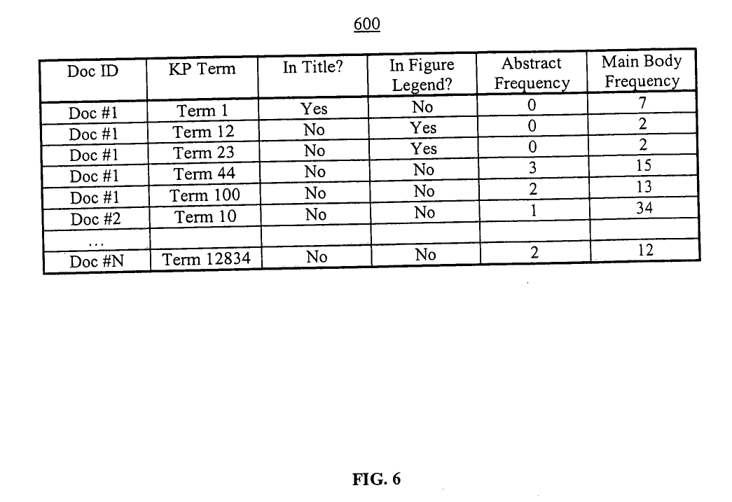

In step 508, spider stores the information acquired in step 506 into document database 146. FIG. 6 illustrates an example database table 600 that can be used to store the information. As shown in FIG. 6, table 600 includes a number of rows with each row having six fields: a document-ID field 601 for storing a document identifier, a knowledge pack word (KPW) field 602 for storing a word from the knowledge pack 122, a document-title field 603 for storing an indication of whether the word in the KPW field 604 appears in the title of the document identified by the document identifier, a figure legend field 604 for storing an indication of whether the word in the KPW field 104 appears in the a figure legend of the document, an abstract field 605 for storing a value that corresponds to the number of times the word in the KPW field 602 appears in the documents abstract; and a main body field 606 for storing a value that corresponds to the number of times the word in the KPW field 602 appears in the main body of the document.

As shown in the example table 600, there are only five words from the KP 122 in doc1. That is, doc1 includes the following words form the KP 122: word1, word2, word3, word4 and word5. As table 600 informs us, only word1 appears in the tile of the doc1 and only word2 and word3 appear in a figure legend. Table 600 also informs us that word4 appears 3 times in the abstract and 15 times in the main body of the document.

In step 510, spider 144 determines whether there are more words in the KP 122. If so, the process returns to step 504 where spider 144 selects a new word or term from the KP 122, otherwise the process continues to step 512. In step 512, spider 144 determines whether there are more documents that need parsing. If so, the process returns to step 502, otherwise the process may end.

By creating database 146, Xactans 100 can determine the above mentioned frequencies without having to retrieve all of the documents identified in a search result. This feature greatly increases the speed with which Xactans 100 scores the documents identified in a search result.

As mentioned above, Xactans 100, in some embodiments, uses the frequency information to assign a score to each document. In these embodiments, Xactans 100 includes a scoring module 150 for this purpose. In some embodiments, module 150 implements a scheme of relationship scoring through a network of relational matrices in order to determine the score of a document. Each matrix in the network is used to score data based on particular criteria, such as proximity to the query term and the number of exact matches, proximity and frequency of synonyms, the location of these terms in the document-i.e. in the title, abstract or body of the text.

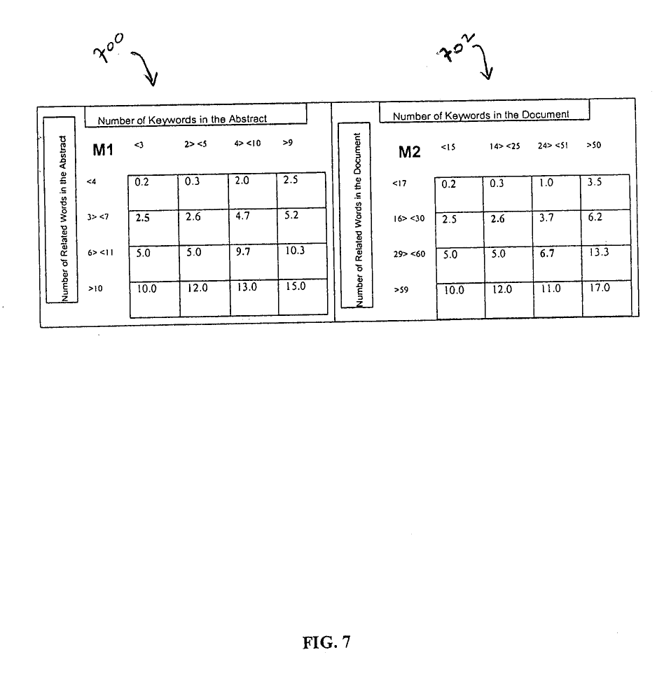

In addition, the network may include a matrix that shows relationship between a keyword and its synonyms and/or related words. For example, the number of times a keyword is found in the abstract may be associated with a number times the keyword's synonyms and/or related terms are found in the abstract, such that an instance of the matrix element would produce a specific score. This is represented in FIG. 7.

FIG. 7 shows a two dimensional matrix 700 that is used in scoring a document. Each cell of matrix 700 is associated with a particular pair of frequencies and each cell has a value, thus the value is associated with a particular pair of frequencies. For example, matrix 700 provides a score given the number of keywords in the document's abstract and given the number of related words in the abstract. As a specific example, if we counted 4 keywords in the abstract and 11 related words in the abstract, then matrix 700 indicates that the score for this scenario should be 12.0. This score can be added or otherwise combined with other scores determined from other matrices, such as matrix 702, to determine the total score for the document.

The previous example could also be associated with a number of related words in the same paragraph, yielding a three dimensional matrix with three relationships. A software routine or routines would run parameters against available matrices to come up with a partial score for each matrix. The total score of the matrix is always constant, but element scores within any matrix are dynamic statistical probabilities of occurrences and change through a feedback mechanism. The presented approach is a slight modification of a Markov Model shown here: P(total)=P(x₁)P(x₂|x₁)P(x₃|x₂) . . . P(x_{L}|x_{L-1}), where P(total) is the product of individual probabilities P(x) for a total of L number of instances.

In Markov's model the total probability is the product of individual probabilities where each unique occurrence in a system is associated with a specific probability that can be adjusted through training of a system. In systems according to the present invention, initial values in the matrix are arbitrary probabilities derived from an initial dataset. Software feedback will use the algorithm below to adjust individual probabilities in the matrix as more data is processed: P(x_{cell})=(adjustment_{cell})*(x_{cell}/Σx_{cell}).

All other matrices in the matrix network would have an associated score for a particular set of frequency data. The scores from each matrix would then be added to produce a total score. The scores may be added up in the same way as impedance in an electrical circuit. A total score would represent a total assessment of all the relationships in our model. Based on user preferences, a feedback mechanism would be able to weight adjust each matrix's output based on search profile input. This user induced feedback method, upon execution, will allow for fine-tuning of the selectivity of the query results.

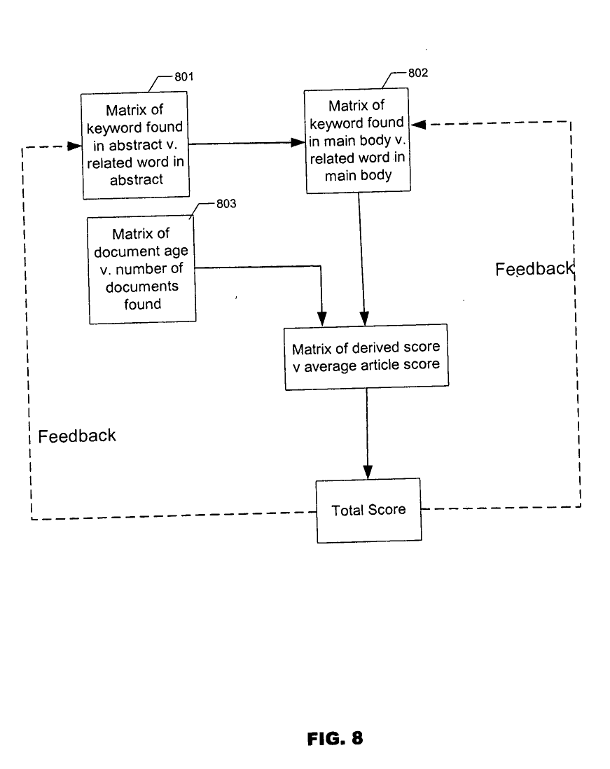

FIG. 8 illustrates an example matrix network. Matrices configured in series would require an input from a previous matrix's output, thus establishing a sequential relationship (e.g., matrix 802 requires an input from matrix 801). Parallel matrices (e.g., matrices 801 and 803) would be independent of each other's output and could process information concurrently. The scoring process could be distributed by using multithreaded logic of parallel processing as opposed to sequential processing of serial logic data. As stated above, adding matrix scores in parallel would be different than adding scores in series, where the serial dependent relationship, consisting of more than one dependent step, produces a higher total score than for independent matrices in parallel.

A software array, which can be multidimensional, could be used to represent each matrix, and thus the relationship model can be easily modified in terms of software development and updates. During execution, array data that represents a score for a relational instance could be adjusted through a software feedback mechanism. In some embodiments, the Java programming language is used to implement some or all of scoring module 150. Java is a powerful programming language for working with arrays and matrices, since many methods have already been implemented that would simplify the development process. Java is also operating system agnostic and thus allows for greater flexibility for development and execution.

In a more specific scenario of how a document would be scored, a specific number would be generated for each parameter of interest during a parsing of each retrieved document. As discussed above, parameters of interest include the number of times certain words or terms appear in different sections of the document. The scoring module, however, could also use additional parameters for each document, such as the age of the document, overall number of documents found as a result of the search, the publisher of the document, etc. Each parameter can be given a default weight so that some parameters influence the total score more than others. Xactans 100, however, is designed so that the weights can be easily modified as it is important to structure the program such that it can be easily altered and parameter structures modified. Scores for all matrices would then be added up to generate a total score. The total score of perceived relevance that is generated along with the document identifier may be passed back to query module 120, which would process and present results to the end user.

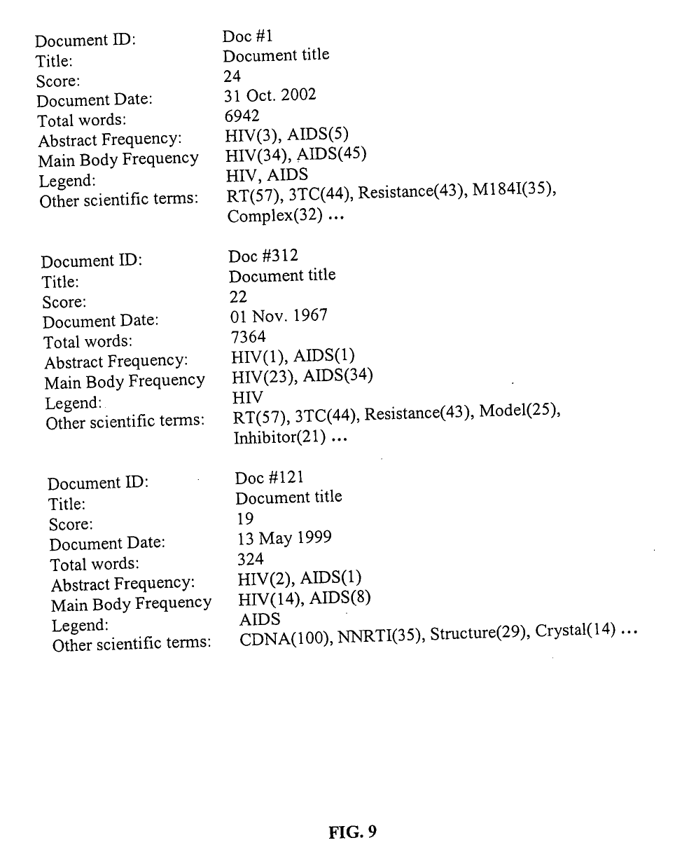

FIG. 9 illustrates an example output that is presented to user 101 after a search has been completed and the resulting documents have been scored. In the example shown in FIG. 9, user 101's initial query was “HIV” and user 101 selected AIDS as a related word. Thus, the final query was “HIV” or “AIDS”. As shown in FIG. 9, the documents are presented in decreasing order of score so that the highest scoring document is presented at the top of the list and the lowest scoring document is presented at the bottom of the list. As also illustrated in FIG. 9, a variety of information may be presented to the user. For instance, for each document, Xactans 100 may display the document's identifier (e.g., URL or title), the document's title (if the title is not used as the document's identifier), the score of the document, and statistical and other information. The statistical information may include: (1) the query term abstract frequency; (2) the query term main body frequency; and (3) for each word in the knowledge pack 122 that is found in the document, the frequency with which the word appears in the abstract and main body (or simply the total frequency—abstract frequency plus main body frequency). The other information may include information regarding whether a query term was found in a figure legend. Advantageously, user 101 may request that Xactans 100 save the results of the search for later retrieval by activating the a save button (not shown) (step 222).

As shown in FIG. 9, with respect to the first document in the list (i.e., the highest scoring document): (a) the term HIV was found twice in the abstract and AIDS was found three times in the abstract; (b) the term HIV was found 34 times in the main body of the document and the term AIDS was found 45 times in the main body; (c) both terms HIV and AIDS appeared in a figure legend; and (d) terms from the knowledge pack 122 that appeared in the document include: RT (appearing 57 times), 3TC (appearing 44 times), Resistance (appearing 43 times), M184I(appearing 35 times), and Complex(appearing 32 times). Accordingly, the output of Xactans 100 provides a great deal of information that enables user 101 to quickly and easily find the information for which the user is searching.

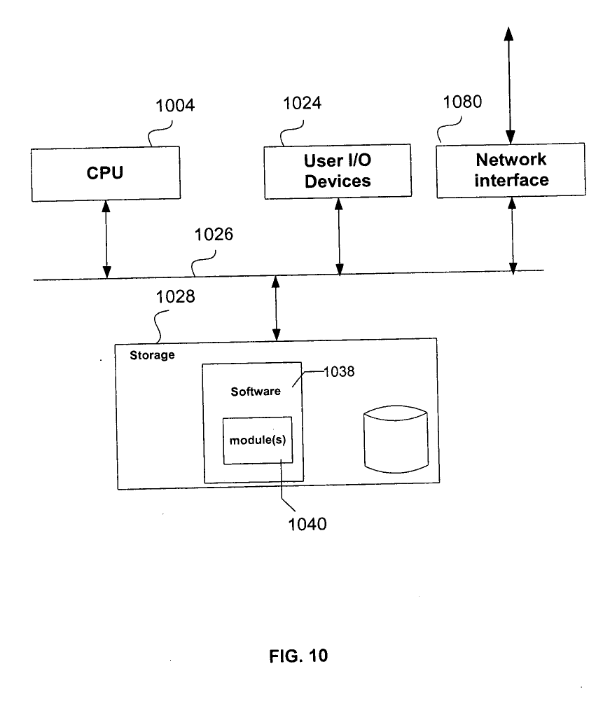

FIG. 10 is an illustration of a representative computer system 1000 that can be used to implement the systems and methods (or components or steps thereof) of the present invention. Computer system 1000 includes a processor or central processing unit 1004, such as, for example, an Intel-based CPU capable of executing a conventional operating systems central processing unit 1004 communicates with a set of one or more user input/output (I/O) devices 1024 over a bus 1026 or other communication path. The I/O devices 1024 may include a keyboard, mouse, video monitor, printer, etc. The CPU 1004 also communicates with a computer readable medium (e.g., conventional volatile or non-volatile data storage devices) 1028 (hereafter “storage 1028”) over the bus 1026. The interaction between CPU 1004, I/O devices 1024, bus 1026, network interface 1080, and storage 1028 are well known in the art.

Storage 1028 can store one or more of the databases discussed above. Storage 1028 may also store software 1038. Software 1038 may include one or more software modules 1040 for implementing the modules discussed above. Conventional programming techniques may be used to implement these modules. Storage 1028 can also store any necessary data files.

In addition, computer system 1000 may be communicatively coupled to the Internet and/or other computer network through a network interface 1080 to facilitate data transfer and operator control.

The systems, processes, and components set forth in the present description may be implemented using one or more general purpose computers, microprocessors, or the like programmed according to the teachings of the present specification, as will be appreciated by those skilled in the relevant art(s). Appropriate software coding can readily be prepared by skilled programmers based on the teachings of the present disclosure, as will be apparent to those skilled in the relevant art(s). The present invention thus also includes a computer-based product which may be hosted on a storage medium and include instructions that can be used to program a computer to perform a process in accordance with the present invention. The storage medium can include, but is not limited to, any type of disk including a floppy disk, optical disk, CDROM, magneto-optical disk, ROMs, RAMs, EPROMS, EEPROMS, flash memory, magnetic or optical cards, or any type of media suitable for storing electronic instructions, either locally or remotely.

While the processes described herein have been illustrated as a series or sequence of steps, the steps need not necessarily be performed in the order described, unless indicated otherwise. Also, while the modules of Xactans 100 illustrated in FIG. 1 are shown as being separate entities, they need not be. As will be apparent to those skilled in the art of computer programming, a single piece of software or multiple pieces of software can implement the modules. If multiple pieces of software implement the modules, the pieces do not need to run on the same computer.

The foregoing has described the principles, embodiments, and modes of operation of the present invention. However, the invention should not be construed as being limited to the particular embodiments described above, as they should be regarded as being illustrative and not as restrictive. It should be appreciated that variations may be made in those embodiments by those skilled in the art without departing from the scope of the present invention. Obviously, numerous modifications and variations of the present invention are possible in light of the above teachings. It is therefore to be understood that the invention may be practiced otherwise than as specifically described herein.

Thus, the breadth and scope of the present invention should not be limited by any of the above-described exemplary embodiments, but should be defined only in accordance with the following claims and their equivalents.

What is claimed is:

1. An information retrieval method, comprising:
prompting a user to input an initial query and receiving the initial query input by the user, wherein the initial query includes a keyword;
determining a synonym of the keyword;
determining a term related to the keyword;
creating a first query, wherein the first query (a) includes the keyword, the synonym, and/or the related term and (b) conforms to the query protocol of a first search engine;
creating a second query, wherein the second query (a) includes the keyword, the synonym, and/or the related term and (b) conforms to the query protocol of a second search engine;
submitting to the first search engine the first query;
submitting to the second search engine the second query;
receiving from the first search engine a first plurality of document identifiers;
receiving from the second search engine a second plurality of document identifies; and
for one or more document identifier included in the first plurality of document identifiers and for one or more document identifier included in the second plurality of document identifiers, determining a score for the document identified by the document identifier,
wherein the step of determining the score includes the step of identifying a figure legend within the document, and
wherein the document's score is, at the least, a function of whether the keyword, synonym and/or related word is found in the identified figure legend.

2. The method of claim 1, further comprising the step of enabling the user to select the synonym, wherein, if the user selects the synonym, then the first query includes both the keyword and synonym.

3. The method of claim 1, further comprising the step of enabling the user to select the related term, wherein, if the user selects the related term, then the first query includes both the keyword and related term.

4. The method of claim 1, wherein the first query include the keyword but not the synonym or related term.

5. The method of claim 4, further comprising the steps of:
creating a third query, wherein the third query (a) includes the synonym, but not the related term or the keyword and (b) conforms to the query protocol of a first search engine;
submitting to the first search engine the third query; and
receiving from the first search engine a third plurality of document identifiers.

6. The method of claim 1, further comprising the step of enabling the user to assign a weight value to the synonym, the related term and/or the keyword.

7. The method of claim 1, wherein the step of determining the synonym includes the step of searching for the keyword within a knowledge pack.

8. The method of claim 1, wherein the step of determining a score for a document includes the step of determining the number of times the keyword appears in an abstract of the document and determining the number of times the keyword appears in a main body of the document.

9. The method of claim 8, wherein the step of determining the number of times the keyword appears in the abstract of the document includes the step of accessing a document database that stores statistical information about the document, including the number of times a word in a knowledge pack appears in the document's abstract and main body.

10. The method of claim 8, wherein the step of determining the number of times the keyword appears in the abstract of the document includes the steps of:
retrieving the document after submitting the queries to the search engines; and
parsing the document after retrieving the document.

11. An information retrieval system, comprising:
means for prompting a user to input an initial query;
means for receiving the initial query input by the user, wherein the initial query includes a keyword;
means for determining a synonym of the keyword;
means for determining a term related to the keyword;
means for creating a first query, wherein the first query (a) includes the keyword, the synonym, and/or the related term and (b) conforms to the query protocol of a first search engine;
means for creating a second query, wherein the second query (a) includes the keyword, the synonym, and/or the related term and (b) conforms to the query protocol of a second search engine;
means for submitting to the first search engine the first query;
means for submitting to the second search engine the second query;
means for receiving from the first search engine a first plurality of document identifiers;
means for receiving from the second search engine a second plurality of document identifies; and
scoring means for determining a score for a document identified by a document identifier from the first or second plurality of document identifiers, the scoring means including means for identifying a figure legend within the document, wherein the document's score is, at the least, a function of whether the keyword, synonym and/or related word is found in the identified figure legend.

12. The system of claim 11, further comprising means for enabling the user to select the synonym, wherein, if the user selects the synonym, then the first query includes both the keyword and synonym.

13. The system of claim 11, further comprising means for enabling the user to select the related term, wherein, if the user selects the related term, then the first query includes both the keyword and related term.

14. The system of claim 11, wherein the first query include the keyword but not the synonym or related term.

15. The system of claim 14, further comprising:
means for creating a third query, wherein the third query (a) includes the synonym, but not the related term or the keyword and (b) conforms to the query protocol of a first search engine;
means for submitting to the first search engine the third query; and
means for receiving from the first search engine a third plurality of document identifiers.

16. The system of claim 11, further comprising means for enabling the user to assign a weight value to the synonym, the related term and/or the keyword.

17. The system of claim 11, wherein the means for determining the synonym includes means for searching for the keyword within a knowledge pack.

18. The system of claim 11, wherein the scoring means includes means for determining the number of times the keyword appears in an abstract of the document and mean for determining the number of times the keyword appears in a main body of the document.

19. The system of claim 18, wherein the means for determining the number of times the keyword appears in the abstract of the document includes means for accessing a document database that stores statistical information about the document, including the number of times a word in a knowledge pack appears in the document's abstract and main body.

20. The system of claim 18, wherein the means for determining the number of times the keyword appears in the abstract of the document includes:
means retrieving the document after submitting the queries to the search engines; and
means for parsing the document after retrieving the document.

21. A computer program embodied on a computer readable medium, the computer program comprising:
a computer code segment for prompting a user to input an initial query;
a computer code segment for receiving the initial query input by the user, wherein the initial query includes a keyword;
a computer code segment for determining a synonym of the keyword;
a computer code segment for determining a term related to the keyword;
a computer code segment for creating a first query, wherein the first query (a) includes the keyword, the synonym, and/or the related term and (b) conforms to the query protocol of a first search engine;
a computer code segment for creating a second query, wherein the second query (a) includes the keyword, the synonym, and/or the related term and (b) conforms to the query protocol of a second search engine;
a computer code segment for submitting to the first search engine the first query;
a computer code segment for submitting to the second search engine the second query;
a computer code segment for receiving from the first search engine a first plurality of document identifiers;
a computer code segment for receiving from the second search engine a second plurality of document identifies; and
a computer code segment for determining a score for a document identified by a document identifier from the first or second plurality of document identifiers, said computer code segment including code for identifying a figure legend within the document, wherein the document's score is, at the least, a function of whether the keyword, synonym and/or related word is found in the identified figure legend.

22. The system of claim 21, further comprising a computer code segment for enabling the user to select the synonym, wherein, if the user selects the synonym, then the first query includes both the keyword and synonym.

23. The system of claim 21, further comprising a computer code segment for enabling the user to select the related term, wherein, if the user selects the related term, then the first query includes both the keyword and related term.

24. The system of claim 21, wherein the first query includes the keyword but not the synonym or related term.

25. The system of claim 24, further comprising:
a computer code segment for creating a third query, wherein the third query (a) includes the synonym, but not the related term or the keyword and (b) conforms to the query protocol of a first search engine;
a computer code segment for submitting to the first search engine the third query; and
a computer code segment for receiving from the first search engine a third plurality of document identifiers.

26. The system of claim 21, further comprising a computer code segment for enabling the user to assign a weight value to the synonym, the related term and/or the keyword.

27. The system of claim 21, wherein the computer code segment for determining the synonym includes code for searching for the keyword within a knowledge pack.

28. The system of claim 21, wherein the computer code segment for determining a score for the document includes code for determining the number of times the keyword appears in an abstract of the document and code for determining the number of times the keyword appears in a main body of the document.

29. The system of claim 28, wherein the code for determining the number of times the keyword appears in the abstract of the document includes code for accessing a document database that stores statistical information about the document, including the number of times a word in a knowledge pack appears in the document's abstract and main body.

30. The system of claim 28, wherein the code for determining the number of times the keyword appears in the abstract of the document includes:
a computer code segment for retrieving the document after submitting the queries to the search engines; and
a computer code segment for parsing the document after retrieving the document.
